(19) United States
(12) Patent Application Publication
(10) Pub. No.: US 2002/0198429 A1
(43) Pub. Date: Dec. 26, 2002

(54) Inducing turbulent flow in catalyst beds

(75) Inventors: Sriram Ramani, Ponca City, OK (US); Joe D. Allison, Ponca City, OK (US); Richard Delgado, Ponca City, OK (US)

Correspondence Address: DAVID W. WESTPHAL, CONOCO PHILLIPS, P.O. BOX 4783, HOUSTON, TX, 77210-4783, US

(73) Assignee: Conoco Inc., Houston, TX (US)

(21) Appl. No.: 10/150,850

(22) Filed: May 16, 2002

Related U.S. Application Data
(60) Provisional application No. 60/292,033, filed on May 18, 2001.

(51) Int. Cl. C07C 5/367; C07C 5/333; B01J 35/02

(52) U.S. Cl. 585/658; 585/661; 422/211; 422/222

(57) ABSTRACT

A catalyst bed is made of a monolith having a plurality of pores extending through the monolith, the pores forming tortuous flow paths through the monolith. The tortuous flow paths are obtained by modifying the monolith channels with turbulence-inducing objects or means. Catalyst is disposed on the wall surfaces formed by the pores. Reactants are passed through the tortuous flow paths creating turbulent flow thereby increasing the contact of the reactants with the catalyst on the wall surfaces and the mixing across the reactant stream.

### CROSS-REFERENCE TO RELATED APPLICATIONS

This application claims the benefit under 35 U.S.C. §119(e) of U.S. Provisional Patent Application No. 60/292,033 filed May 18, 2001.

### STATEMENT REGARDING FEDERALLY SPONSORED RESEARCH OR DEVELOPMENT

Not applicable.

### FIELD OF THE INVENTION

The present invention generally relates to catalyst beds in a reaction system having a substrate, and more particularly to a catalyst substrate having flow paths therethrough, which induce turbulent flow providing increased mixing and reaction time of the reactants flowing through the catalyst bed, and still more particularly to a honeycomb monolith having pores therethrough for inducing turbulent flow to allow increased mixing and reaction time of a syngas or Fischer-Tropsch or olefins reactant stream while minimizing the pressure drop of the reactant stream across the honeycomb monolith.

### BACKGROUND OF THE INVENTION

Reactant flow through a reaction system is of critical importance including flow over and through the catalyst bed or zone as well as throughout the entire process. Flow of a reactant stream has many facets that must be carefully balanced in order to optimize a reaction system and make the system most economical and safe.

In many industrial processes, a pressurized flow of a reactant stream is provided in order to increase the production output and maximize the economics of the operation. The catalyst zone of a reactor is one of the principal stages of the process. In the catalyst zone, catalysts are generally introduced into the system as a coating on a stationary or fixed substrate in the flow pathway of the reactants. These substrates must provide enough surface area so that a sufficient amount of catalyst may be available to the reactants, as well as sufficient amount of void space in order to minimize the pressure drop across the catalyst bed. Pressure drops within a system are undesirable because they require an increase of energy into the system.

It is preferred that the catalyst bed maximizes the contact time of the reactants with the catalyst while still minimizing the pressure drop across the catalyst bed. Contact time is the number and frequency of contact between the reactant molecules and the catalyst as the reactants pass through the honeycomb monolith. To state it another way, contact time is a measure of collisions between the reactants and catalyst before the reactants exit the catalyst bed. The actual residence time of the reactants within the catalyst bed is not as important as the frequency and number of times the reactant molecules contact the catalyst surface. This is beneficial to the reaction in two ways. First, an increase in the frequency of collision between a particular reactant molecule with catalyst atoms increases the probability that the reactant molecule will react or undergo the desired chemical change. Second, an increase in the number of collisions between unreacted reactant molecules and catalyst increases the probability that most of the reactant stream molecules will have an opportunity to react. Thus, an increased number of collisions between reactant molecules and catalyst produces a higher yield of desired product.

Turbulence increases the frequency of collision of the reactant molecules with the catalyst and therefore one method of increasing contact is inducing turbulent flow of the reactants through the catalyst bed. Turbulence causes the reactant molecules to move in a random manner, colliding with themselves as well as contacting the catalyst. The chaotic movement of the reactant molecules is desirable because it not only increases contact with the catalyst but also increases mixing. Enhanced mixing increases the contact of the reactants with the catalyst. By increasing the turbulence of the reactant stream, mixing across the reactant stream is improved to allow a higher frequency or chance of collision of an unreacted reactant molecule and a catalyst. Thus an objective is to induce turbulence, without substantially affecting adversely the pressure drop across the catalyst bed, to maximize the frequency of contact between the reactants and catalyst thus providing an improved and more complete reaction. The impact of turbulence on the chemical reaction will vary with the type of catalyst being used, but without regard to the type of catalyst being used, turbulence will enhance the chemical reaction.

Typically a higher flow rate will induce greater turbulence. A lower flow rate tends to cause the flow to be more laminar. If there is no pressure drop, then there should be no reduction in the flow rate of reactants through the catalyst bed. Reducing flow may result in more residence time between the reactants and catalyst but lower flow rates decrease the production rate in a process. In general, it is always desirable to increase the production rate and therefore it is preferred to maximize the flow rate through the catalyst bed to the extent possible.

Prior art substrates for these systems have taken various forms. One of the more conventional substrates has been the use of straight channel beds through the catalyst, such as the honeycomb monolith where the reactants pass through the straight channels. The channels may be circular in cross-section or may be square or rectangular in cross-section. A catalyst bed having straight channels is typically a honeycomb monolith with a plurality of straight channels passing through it. The monolith may be of any desired dimensions depending on the particular reaction system with the walls of the channels being perpendicular to the top and bottom of the honeycomb monolith.

A straight channel flow path provides a high amount of surface area and a high amount of void space with an almost negligible pressure drop. However, straight channels tend to produce laminar flow across the catalyst zone decreasing the output for a given catalyst reaction.

For example, the reactant molecules must contact the catalyst material in order for the reactant molecules to form product. In laminar flow systems only the outer portion of the flow stream contacts the catalytic material coated on the substrate surfaces forming the channels through the catalyst bed. Diffusion of unreacted reactant molecules to the outer portion of the stream (either pressurized or at lower pressure) is limited and without a sufficient amount of mechanical mixing of the stream, the inner portion of the laminar stream of unreacted reactants does not contact the catalyst, thus reducing the extent of reaction and the amount of product produced by the chemical reaction. Once the stream exits the catalyst zone the chance for reaction is gone leaving a large portion of the reactants unreacted.

Examples of chemical processes in which a catalyst bed of the present invention can be used is in syngas or Fischer-Tropsch or olefins reactors. Fischer-Tropsch reactors typically use fluidized catalyst beds, but stationary beds are not uncommon. For purposes of clarity and illustration only, a syngas reaction system will be discussed herein. It should be understood that the present invention should not be interpreted and is not contemplated as limited to only syngas or Fischer-Tropsch reactors, but instead, is wholly appropriate and useful for any system where increasing the turbulent flow of a gas or liquid is desirable. In a typical syngas reactor, catalyst is coated onto a honeycomb straight channel monolith. Honeycomb monolithic substrates are typically constructed from an extruded ceramic refractory material such as cordierite (2MgO-5SiO₂-2Al₂O₃) or mullite (3Al₂O₃-2SiO₂), usually as a cylinder or disk, although any shape can be extruded as necessary for a certain application. Formed metallic foil monolithic structures are also commonly used. The size of the honeycomb monolith will vary with the chemical reaction and catalyst. However, flow is predominately laminar throughout the monolith causing a lower efficiency and reduced yield of the chemical reaction.

One attempt to produce turbulent flow through a straight channel or honeycomb type bed has been to dispose objects, such as baffles, into the channels to provide turbulence and mixing throughout the length of the catalyst bed thereby allowing for the desired increase in the frequency of collisions between the reactants and catalyst. However, the straight channels have a very small diameter, usually 1 to 2 millimeters or less, providing very limited space through the channels for baffles. Further the channels are extremely numerous. Disposing baffles in each of these small channels greatly increases the pressure drop across the catalyst bed. In addition, it is difficult to manufacture consistent baffles at that scale. This leads to inconsistency in the catalyst coating of the bed and can generate hot spots, unpredictable production results, and pressure gradients across the bed.

Another attempt to overcome the inherent problems of the straight channel or honeycomb type bed is to induce mixing and turbulence in the flowing reactant stream prior to the stream entering the catalyst bed. This turbulent flow mixes the reactants and directs the turbulent flow of the reactants into the straight channels of the honeycomb monolith. The laminar flow is interrupted and the stream enters the catalyst bed in a turbulent state. Thus, the reactants do not pass into the straight channels with a laminar flow but instead with a turbulent flow with the purpose of enhancing the chemical reaction, i.e., the contact of the reactants with the catalyst.

Although mixing of the stream is increased, the turbulence is short lived. Due to the straight channels of the catalyst bed, the flow of the stream generally becomes laminar quickly after leaving the mixing zone. Similarly simply increasing the pressure of the reactant stream can increase turbulence, but the flow is forced to become laminar quickly within the channels.

An alternative to the straight channel design is a packed catalyst bed that attempts to balance the pressure drop and increased turbulence. These kinds of packed beds are well known in the art and are numerous in variation.

One common type of a packed bed is a packed bed of spheres or cylinders. The catalyst is pre-coated onto the spheres or cylinders and the coated spheres or cylinders are then packed into a column. The reactant stream flows through the winding flow paths formed around the spheres or cylinders as it passes through the catalyst bed thus creating turbulence and enhancing the catalytic reaction. The void space between the packed spheres or cylinders in these beds is typically smaller than the non-void space, thus creating a high surface area bed. The size of the spheres or cylinders can be manufactured uniformly. This uniformity allows for greater control of the concentration of catalyst within the bed. Knowledge of the catalyst concentration allows predictability of the amount of product that can be produced for a given reaction. Although there is some control and predictability in this type of bed the pressure drop is higher due to the decreased amount of void space compared with that of the straight channel structures.

Still another method is to dispose an object in the path of the flowing reactants to induce turbulence. One method of introducing foreign objects in the reactant flow path includes cutting the monolith into sections and introducing a foreign object between the sections at the channels such that the reactants must flow around or through the foreign objects as the reactants flow through the channels thus creating turbulence and additional contact with the catalyst surface. One type of foreign object to place is a gauze or wire mesh in between the adjacent sections of the monolith. The gauze or wire mesh size is smaller than the diameter or width of the channel. The wire could also be coated with catalyst. The reactants impinge upon the wire, breaking up the laminar flow through the channels, thus creating turbulence as the reactants pass through the channels. Further, the wire mesh may have different densities so that the particular size mesh of the wire can be varied, thus varying the density of the mesh or foreign matter between the sections of honeycomb monolith.

The wire gauze or mesh is typically preferred due to its low pressure drop and control of catalyst concentration. However, the reaction temperatures in the catalyst zone are typically high, approaching 2500° F., which require the gauze or mesh to be made from special metal alloys to withstand such temperatures and accordingly are very expensive. Thus, using a fixed bed comprised entirely of gauze or mesh may be cost prohibitive. The gauze or mesh can be manufactured to a particular density and thus has predictability. However, when the gauze or mesh is placed in a stacked bed, the orientation of the sheets is not consistent. Although one could attempt to align all of the wire strands making up the gauze or mesh, it is highly impractical in an industry setting. The pressure drop across gauze or mesh is less than in a packed bed of spheres or cylinders but higher than that of foam and straight channels. The pathway in gauze or mesh is more random than the packed spheres or straight channels but less random than foam. Another disadvantage to using a fixed bed comprised entirely of gauze or wire mesh is the relative ability for collisions by the reactants on the low surface area of the wire, although the expense is generally considered the major drawback.

One type of catalyst bed includes a three-dimensional foam structure. Foam has the advantage of having a negligible pressure drop due to the fact that the foam is predominately void space, i.e., typically around 80%. However, this large void space causes foam substrates to have the least amount of surface area for catalyst deposition. Pressure drop is measured in inches of water column. The pressure drop may be 5 to 10 inches of water column across a foam structure with flow rates of approximately 10 feet per second. Also the foam with its variable pores creates turbulent flow of the reactants.

Foam substrates have a random porosity during production due to their manufacturing process. These variances make it difficult to calculate the surface area of each foam substrate. Although the cell size through the foam is within a range, the different sizes of the cells through the foam does vary. This range is wide enough and the manufacturing process is variable enough that the foam produces too wide a physical property range to be desirable. Thus, the foam provides a somewhat unpredictable catalytic process. The varied distribution of pore sizes through the foam and the varied distribution of pore orientations and the variable distribution of pore shapes causes the foam to be unpredictable. The distribution of the catalyst on the surfaces formed by the foam pores is very unpredictable thus causing the foam to provide an uneven catalytic coating whereby the amount of chemical reaction which will be achieved is unpredictable.

The pressure drop across a packed catalyst bed is traditionally substantially higher than that of foams, possibly 3 to 5 times the pressure drop across foams. In contrast to a foam substrate which typically has greater than 80% void space, a packed catalyst bed of spheres, with sphere sizes that approximate the opening cell size, has only 30% void space. Thus, the surface area in a packed bed is substantially greater than the surface area provided by a foam structure.

Therefore, there is still a need to produce a substrate for a reaction system that can achieve increased turbulence and mixing of the flowing reactant stream with a low or negligible pressure drop. The present invention overcomes the deficiencies of the prior art while focusing on these needs.

### BRIEF SUMMARY OF THE INVENTION

A catalyst bed consisting of a monolith having a plurality of pores extending through the monolith, the pores forming tortuous flow paths through the monolith. Catalyst is disposed on the wall surfaces formed by the pores. Reactants are passed through the tortuous flow paths creating turbulent flow thereby increasing the contact of the reactants with the catalyst on the wall surfaces and the mixing of the reactants.

The present invention is a turbulence inducing substrate to be used in the preferred embodiment as a catalytically active fixed bed in a reaction system with a flowing reactant stream. The primary feature of the present invention is that the turbulent flow of the reactants causes an increase in mixing within the reactant stream and increased frequency of collisions between the reactants and the catalyst with only a negligible pressure drop across the catalyst bed.

Turbulence in the preferred embodiment is achieved by creating a plurality of flow channels through the monolith structure each having a tortuous path therethrough for the turbulent flow of reactants. Although the present invention could encompass a single flow channel, a plurality of channels is preferred due to the increase in total surface area for catalyst deposition. The substrate structure of the preferred embodiment is a modified honeycomb monolith coated with a catalytic composition. The present invention may also include additional turbulence inducing materials in combination with a modified honeycomb monolith.

In the preferred embodiment, a modified honeycomb type structure is used to take advantage of its superior pressure characteristics over other types of packing such as a packed bed of spheres or cylinders. The monolith is modified to induce turbulence by creating an irregular pathway for flow of the reactants through the modified monolith as compared with the typical straight channel pathway of a honeycomb monolith.

In accordance with the present invention, modification of a honeycomb monolith can be accomplished in a variety of ways. One embodiment is to manufacture a single integral unit with a modified pathway. Honeycomb monoliths are typically constructed from ceramic material that can be extruded in a predetermined honeycomb design. The term “honeycomb” comes from the plurality of channels that form the flow pathways extending axially through the monolith such that they closely resemble that of an actual honeycomb.

One way is to rotate or twist the wet extrudate during extrusion at various time intervals causing a spiraling of the channel flow pathways. As the rotations become more frequent, approaching infinity, the walls of the pathway can produce a continuous surface, spiraled pathway through the monolith. In other words, as the transitions between the rotated and non-rotated sections become smaller and smaller the angles or bends in the pathways become less apparent. This effect on the pathway wall may be considered “smooth.” However, “smooth” should be defined for the present invention as a wall where the transitions have become so close together as to produce a continuous curve or practically continuous curve in the pathway wall. The term smooth as applied to the pathway walls of the present invention should not be interpreted to mean that the texture of the walls is smooth as in reduced friction, since the preferred embodiment may produce a smooth curve with a rough or porous surface. In the most preferred embodiment of the present invention, the time intervals may be more spaced to produce a staggered or stepped wall pathway. The “steps” cause a redirection of the reactant flow increasing turbulence and mixing within the stream.

Alternatively, the wet extrusion product can be sliced into sections. The sections are then stacked together such that channels of each section of the monolith are misaligned. At each of the interfaces where the misalignment occurs, a “step” will result causing a redirection of the reactant flow stream. In yet another embodiment, additional packing elements such as gauze or mire mesh can be introduced by placing them in between the sections of the monolith.

Thus, the preferred embodiment is a substrate comprised of a single integral unit, similar sections of a honeycomb monolith packed together to form an integral unit, or similar sections of a honeycomb monolith packed together with foreign objects to form an integral unit. The modified monolith of the present invention inherently causes a more turbulent flow of a reactant stream through its modified channels causing an increased frequency of collisions between reactants and the substrate surfaces with only a negligible pressure drop across the reaction zone.

In summary, the invention can be described at least the following preferred embodiments. First, a catalyst bed comprising: a monolith, a plurality of pores extending through the monolith, wherein said pores form tortuous flow paths through the monolith by creating or modifying the pores with turbulence-inducing objects or means. This type of catalyst bed would have flow paths which have a tortuosity greater than 1.0. The flow paths may be formed by rotating a wet extrudate of the monolith while it is being produced. Alternatively, the extrudate may be rotated at timed intervals while it is being produced.

Second, a catalyst bed comprising: a plurality of monolith sections each having a plurality of pores extending therethrough, where the pores of one monolith section are misaligned with the pores of an adjacent section to form the tortuous flow paths. The flow paths having a tortuosity of greater than 1.0. The sections could also be misaligned such that the misaligned pores from abutments to flow through the flow paths.

Third a catalyst bed comprising: a plurality of monolith sections and at least one foreign material section disposed between adjacent monolith sections. The material that makes up the foreign material section of this type of catalyst bed could be selected from the group of wire gauze, wire mesh, foam or honeycomb structures. This type of catalyst bed would also have flow paths with tortuosity of greater than 1.0.

The invention also is embodied as a method for causing a chemical reaction comprising: disposing a catalyst on walls formed by pores extending through a monolith, the pore forming tortuous flow paths; flowing reactants through the tortuous flow paths; creating turbulent flow as the reactants flow through the tortuous flow paths; engaging the reactants with the catalyst on the pore walls; and reacting the reactants to form a product.

Other objects and advantages of the invention will appear from the following description.

### BRIEF DESCRIPTION OF THE DRAWINGS

For a detailed description of the preferred embodiments of the invention, reference will now be made to the accompanying drawings in which:

FIG. 1 shows a cross-sectional side view of a typical fixed catalyst bed made of a honeycomb type substrate;

FIG. 1A shows an enlarged two-dimensional view of a particular flow channel in a honeycomb type substrate;

FIG. 2 shows an enlarged two-dimensional view of a particular flow channel in a honeycomb type substrate in which the flow channel is spiraling due to a twisted monolith structure;

FIG. 3 shows a two-dimensional side view of a honeycomb type flow channel where the monolith has been twisted at various points during the wet extrusion;

FIG. 4 shows a perspective side view of a packed bed comprising a honeycomb type substrate where the monolith has been divided into cross sections and packed together;

FIG. 5 shows a two-dimensional representation of a single channel formed by slightly misaligning alternating sliced sections of a monolith;

FIG. 6 shows a two-dimensional representation of a single channel formed by misaligning alternating sliced sections of a monolith without having independent flow channel type formation;

FIG. 7 shows a top view of a two-dimensional representation of channels formed by randomly rotating sliced sections of a monolith;

FIG. 8 shows a top view of a two dimensional representation of a single channel formed by rotating the sliced sections of a monolith in a continuous direction;

FIG. 9 shows a perspective side view of a packed bed comprising a honeycomb type substrate where the monolith has been divided into cross sections and foreign objects have been stacked alternatively with monolith sections.

### DETAILED DESCRIPTION OF THE PREFERRED EMBODIMENTS

In the description that follows, like parts are marked throughout the specification and drawings with the same reference numerals, respectively. The drawing figures are not necessarily to scale. Certain features of the invention may be shown exaggerated in scale or in somewhat schematic form and some details of conventional elements may not be shown in the interest of clarity and conciseness.

The present invention relates to methods and apparatus for creating turbulent flow of a reactant stream through pores in a catalyst bed thereby enhancing contact between the reactants and catalyst with only a negligible pressure drop across the reaction zone. The present invention is susceptible to embodiments of different forms. There are shown in the drawings, and herein will be described in detail, specific embodiments of the present invention with the understanding that the present disclosure is to be considered an exemplification of the principles of the invention, and is not intended to limit the invention to that illustrated and described herein.

In particular, various embodiments of the present invention provide a number of different constructions of a catalyst structure providing a plurality of tortuous flow paths through the catalyst bed. The embodiments of the present invention also provide methods for enhancing turbulent flow. It is to be fully recognized that the different teachings of the embodiments discussed below may be employed separately or in any suitable combination to produce desired results. Reference to upstream or downstream will be made for purposes of description with upstream meaning the reactants before flowing into the catalyst bed and downstream meaning the products from the reaction or unreacted reactants flowing away from the catalyst bed.

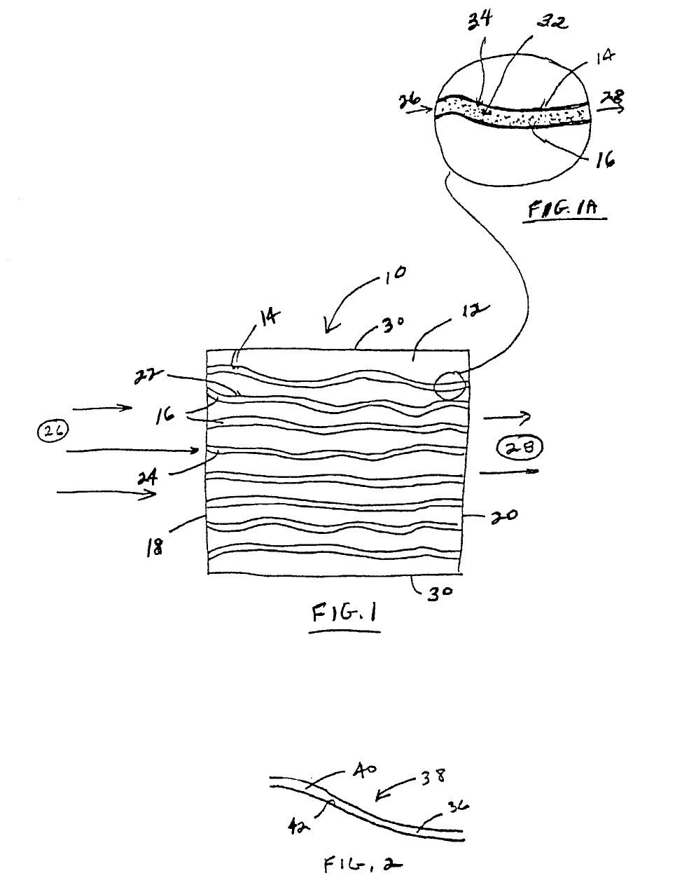

Referring initially to FIG. 1, there is shown a catalyst bed 10, preferably a monolith 12, for supporting a catalyst 14. A monolith is defined as a single piece of substrate made, as for example, of ceramic or other support material. A plurality of pores 16 extends from an upstream end 18 of monolith 12 to a downstream end 20 of monolith 12. A pore is defined as any passage through the catalyst bed 10 extending between the upstream and downstream ends 18, 20.

The cross section of the catalyst bed 10 may be circular or any other shape and cross section that is necessary to fit into a particular reactor. Similarly, the cross section of the individual pores 16 may be circular or any other shape the manufacturing process produces. Circular and square pores are the most common shapes.

Each of the pores 16 form interior wall surfaces 22 creating a tortuous flow path 24 through the monolith 12 for the passage of reactants 26. A tortuous flow path is defined as a flow path that has twists, turns, curves, windings, misalignments, crooks, or any other flow path through the catalyst bed that is not substantially parallel to the sides of the catalyst bed 10.

The tortuous flow paths 24 may be referred to in terms of tortuosity. Tortuosity may be calculated as the ratio of the length of the flow path taken by the fluidized stream flowing through the substrate divided by the length of the shortest straight line path through the substrate, i.e., from the upstream end 18 to the downstream end 20. Thus, a straight channel pathway has a tortuosity of 1.0. The range can increase indefinitely depending on the type and amount of the pores.

In the preferred embodiment, structural changes in the monolith create a tortuous flow path 24, i.e., a flow path with a tortuosity greater than 1.0, for the reactant stream 26 causing the reactants to mix and, thus, allow more of the reactants to come into contact with the catalyst material 14. The honeycomb monolith 12 is preferred due to its low pressure drop character across the catalyst bed 10.

The catalyst 14 is deposited on the wall surfaces 22 of the pores 16, using a number of methods as is well known in the art. After the chemical reaction occurs within the pores 16, product 28 passes from the downstream end 20 of monolith 12. The catalyst is one of the principal components of a catalyzed reaction process. In the catalyst zone, the catalyst 14 is generally introduced into the catalyst bed 10 as a coating on the stationary or fixed wall surfaces 22 forming a catalytically active substrate in the flow pathway 24 of the reactants 26. These substrates must provide enough surface area so that a sufficient amount of catalytic sites 14 may be available to the reactants 26, as well as sufficient amount of void space or flow area through the pores 16 in order to minimize the pressure drop across the catalyst bed 10.

In operation, the reactants 26 enter the upstream end 18 of monolith 12 flowing into the pores 16. The reactants 26 follow the tortuous flow paths 24 impinging on the wall surfaces 22 to contact the catalyst 14 since the flow paths 24 are not parallel with the sides 30 of monolith 12 and the reactants 26 cannot flow through the monolith 12 in laminar flow.

The turbulence caused by the tortuous paths 24 formed by pores 16 increases the frequency of collision of the molecules of the reactants 26 with the catalyst 14 thereby causing the molecules of the reactants 26 to move in a random manner, colliding with themselves as well as contacting the catalyst 14 as they pass through the catalyst bed 10. The chaotic movement of the molecules of the reactants 26 not only increases contact with the catalyst 14 but also enhances mixing which also enhances the contact of the reactants 26 with the catalyst 14. As shown in FIG. 1A, by increasing the turbulence of the reactant stream 26, the interior portion 32 of the reactant stream 26 mixes with the outer portion 34 of the reactant stream 26 to allow a higher frequency or chance of collision of an unreacted reactant molecule and the catalyst surface. Thus turbulence is induced, without substantially affecting adversely the minimum pressure drop across the catalyst bed 10, to maximize the frequency of contact between the reactants 26 and catalyst 14 thus providing an improved and more complete reaction. The impact of turbulence on the chemical reaction will vary with the type of catalyst being used, but without regard to the type of catalyst being used, turbulence will enhance the chemical reaction.

The catalyst bed 10 maximizes the contact time of the reactants 26 with the catalyst 14 while still minimizing the pressure drop across the catalyst bed 10. Contact time is the number and frequency of collisions between the reactant molecules and the catalyst as the reactants pass through the honeycomb monolith.

By way of example and not by way of limitation, the embodiments of the present invention may be used in a reaction system where the catalyst bed is stationary and the reactants are fluidized, such that the reactants are able to flow over or through the catalyst bed. In such chemical processes, reactant molecules must come in contact with a catalyst to initiate a chemical reaction. Catalyst is typically coated onto a substrate of various forms and placed as a stationary or fixed bed in the flow pathway. The most preferred embodiment is designed for a syngas reaction system, but is equally applicable to a Fischer-Tropsch reactor or any reactor where the reactants must pass through a reaction zone.

The honeycomb monolith 12 of the preferred embodiment for a syngas reactor has a thickness between ⅛ and 6 inches. However, the thickness or depth of the honeycomb monolith 12 has no particular limit or size range. The monolith 12 has a plurality of channels or pores 16 that run axially through the substrate providing a large amount of void space. The smaller the width of the pores 16 and the longer the pores 16 are, the greater the need for turbulent flow. The deeper the pores 16, the greater the pressure drop across the catalyst bed 10. Thus, it is not desirable to have unduly long pores 16, since a zero pressure across the catalyst bed 10 is the ultimate goal. Flow through the monolith 12 has a negligible pressure drop.

A tortuous flow path 24 with minimal pressure drop is a preferred objective of the present invention. In the preferred embodiment, the catalyst bed 10 may have a plurality of different flow paths which achieve a tortuosity greater than 1.0.

Referring now to FIG. 2, the tortuous flow paths 36 of pores 40 are produced in honeycomb monolith 38 during the extrusion of the monolith 38 to furnish a winding pathway for the reactant stream 26. During extrusion of the monolith 38, the wet extrudate is continuously rotated or twisted during the extrusion process causing a spiraling of the pores 40 as they are formed. This produces a continuous wall surface 42 of pores 40 and a smooth, even spiraling tortuous flow path 36 through the monolith.

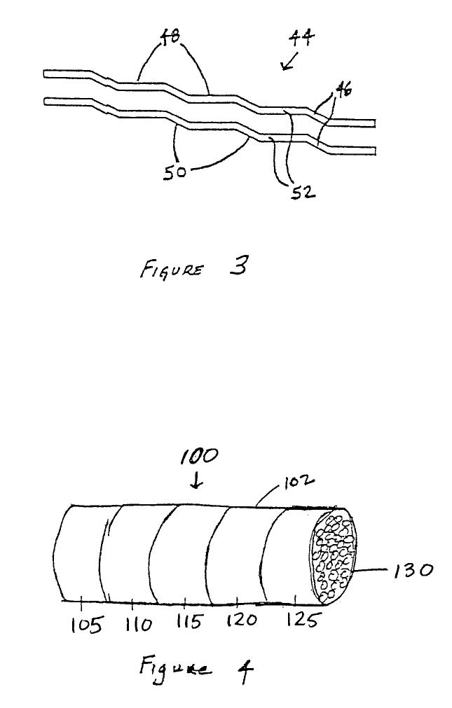

Referring now to FIG. 3, there is shown an alternative method of producing tortuous flow paths during extrusion. During extrusion of the monolith 44, the wet extrudate is rotated or twisted at various time intervals during the extrusion process so as to form a halting spiral of pores 46 as they are formed, assuming the rotation continues in the same direction for the entire extrusion of the monolith. As the rotations become more frequent during extrusion, approaching infinity, the rotations become continuous and the pores 46 become smooth and even such as are shown in FIG. 2. Rotation at time intervals causes a staggered or stepped wall pattern to be formed. As shown in FIG. 3, the pores 46 include straight walled portions 48, and during the rotation periods, angular walled portions 50. The straight walled portions 48 and angular walled portions 50 form “steps” through the pores 46 with tortuous flow paths 52. The “steps” cause a redirection of the reactant flow increasing turbulence or mixing within the stream.

The embodiments of FIGS. 2 and 3 have the advantage of being made from an integral monolith with continuous wall surfaces for reactant flow thus reducing the pressure drop through the flow passages. Further, the monolith is an integral unit that can be reproducibly made, for example, by computer aided mechanical means.

As an alternative to an integral monolith as shown in FIGS. 2 and 3, the monolith need not be integral. Referring now to FIG. 4, there is shown another preferred embodiment of the present invention including a fixed catalyst bed 100 having a sectioned honeycomb monolith 102 that has been produced, such as by cutting, into a plurality of sections 105, 110, 115, 120 and 125 and then stacked to form catalyst bed 100. Each of the sections 105, 110, 115, 120 and 125 may be similar in cross section and axial length, but such is not critical to the present invention. Reproducibility of the catalyst beds between runs in a reactor is an important factor in the industry and it is of importance that sections 105, 110, 115, 120 and 125 be of a practically reproducible length so that predictability can be achieved between beds 100. Each of the sections 105, 110, 115, 120 and 125 may be considered mini-monoliths.

Referring now to FIG. 5, there is shown a top view of a misaligned channel or pore 140 in an arrangement produced by a misalignment of the sections 105, 110, 115, 120 and 125 described in FIG. 1 where the misalignment of adjacent sections 110 and 115, for example, are only slightly off center. Where the walled surfaces 147, 149 are misaligned, a shoulder or abutment 151 is formed causing the reactant stream 145 to impinge against the abutment 151 so as to cause a perturbation in the reactant stream flowing through the misaligned channel 140. The perturbation causes turbulence and results in increased turbulence and mixing of the reactant stream 145.

Referring now to FIG. 6, there is shown an arrangement where adjacent sections 110 and 115 are completely misaligned such that the walled surfaces 112 and 122 bisect the reactant flow pathway 150. The reactant stream 155 impinges against each exposed wall surface creating a tortuous pathway 150 throughout the monolith. In the arrangement shown in FIG. 6, mixing is increased not only from the perturbation caused by the misaligned walls but also from the intermixing of reactant streams from one misaligned pore to another.

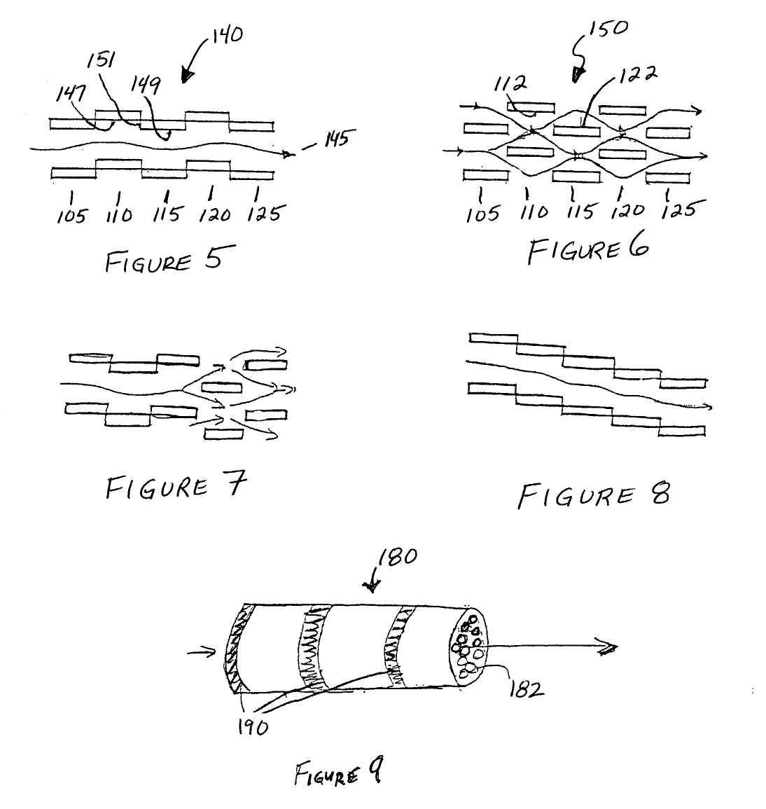

One skilled in the art will appreciate that an infinite number of arrangements can be envisioned by having different numbers of mini-monoliths and varied rotation of the sections to create new patterns in the tortuous pathway. For example, FIG. 7 shows a combination of rotations from FIG. 5 and 6, while FIG. 8 shows a continuous “stepped” wall pattern. The staggered or stepped wall pattern formed, as shown in FIG. 8, is more abrupt than the sharp abutments of FIG. 3. The number of sections is limited by the total length of the monolith or fixed bed in the most preferred embodiment for a syngas reactor.

It should also be appreciated that FIGS. 5, 6, 7 and 8 are two dimensional representations of a top view of a flow path. In actuality, there are three dimensional patterns created by the misalignment of the rotated sections. The three dimensional effect of the pathway shown in FIG. 8 is a spiraling tube with jagged or stepped walls. The reactant stream can be redirected in a three dimensional pattern depending on the available void space or pathway. The redirected portion of the reactant stream is forced to mix with that portion of the reactant stream that is uninhibited by the misaligned walls and becomes part of its forward motion.

The previous preferred embodiments have been produced from the same material for the substrate. Referring now to FIG. 9, there is shown another alternative where an additional material may be used. A sectioned honeycomb monolith is cut into sections 185, similar to those shown in FIG. 4. Sections 185 are stacked together with foreign material sections 190 in between to form a catalyst bed 180. In the preferred embodiment, the foreign material sections 190 may be selected from wire gauze, wire mesh, foam or any combination of these. These foreign material sections 190 furnish increased mixing and turbulence in the reactant flow channels 182 due to their inherent tortuous pathways. In addition, by using thinner sections of the foreign material sections 190, their individual disadvantages, such as cost or hot spots, are minimized.

The make-up of the catalyst bed 180 depends on the desired or necessary conditions of the reaction. If the monolith sections 185 are straight channel sections, several foreign material sections may be necessary to induce sufficient mixing of the reactant stream. As shown, the packed bed 180 of FIG. 9 is stacked as alternating monolith and foreign material sections. The reactant stream 205 initially must go through a foreign material section 190, which would induce turbulence in the stream 205 as it enters the monolith channels 182. The tendency for the stream to develop laminar flow in the channels is reduced as the stream 205 enters the next foreign material section 190, which would again induce mixing and turbulent flow. This phenomenon would be repeated as the reactants continued through the catalyst bed 180.

In an alternative embodiment, the monolith sections could be sliced sections of a modified or twisted monolith as described in FIG. 3. In a packed bed with twisted monolith pores where turbulence is induced by the monolith sections themselves, fewer foreign material sections may be used as additional mixing support. Since the foreign material sections are capable of catalyst on, the entire catalyst bed 180 can be catalytically active.

While preferred embodiments of this invention have been shown and described, modification thereof can be made by one skilled in the art without departing from the spirit or teaching of this invention. The embodiments described herein are exemplary only and are not limiting. Many variations and modifications of the system and apparatus are possible and are within the scope of this invention. Accordingly, the scope of protection is not limited to the embodiments described herein, but is only limited by the claims, which follow, the scope of which shall include all equivalents of the subject matter of the claims.

What is claimed is:

1. A catalyst bed comprising:
a monolith;
a plurality of pores extending through the monolith; and
said pores being modified with turbulence-inducing objects or means so as to form tortuous flow paths through the monolith.

2. The catalyst bed of claim 1 wherein said tortuous flow paths have a tortuosity greater than 1.0.

3. The catalyst bed of claim 1 wherein said tortuous flow paths are formed by rotating a wet extrudate for said monolith as said monolith is being produced.

4. The catalyst bed of claim 1 wherein said tortuous flow paths are formed by rotating at timed intervals a wet extrudate for said monolith as said monolith is being produced.

5. A catalyst bed comprising:
a plurality of monolith sections;
said monolith sections each having a plurality of pores extending therethrough; and
the pores of one monolith section being misaligned with the pores of an adjacent section so as to form tortuous flow paths through the bed.

6. The catalyst bed of claim 5 wherein said tortuous flow paths have a tortuosity greater than 1.0.

7. The catalyst bed of claim 5 wherein said misaligned pores form abutments to flow through said tortuous flow paths.

8. A catalyst bed comprising:
a plurality of monolith sections; and
at least one foreign material section disposed between adjacent monolith sections.

9. The catalyst bed of claim 8 wherein said foreign material section is selected from the group of wire gauze, wire mesh, foam, or honeycomb, wherein such shapes are formed using metal and/or metal oxide compositions, and metals are selected from the transition metal group of elements and metal oxides are selected from the group consisting of ceramic oxide, alkaline oxide or rare earth oxides.

10. The catalyst bed of claim 8 wherein said tortuous flow paths have a tortuosity greater than 1.0.

11. A method for causing a chemical reaction comprising:
(a) disposing catalyst on walls formed by pores extending through a monolith, the pores forming tortuous flow paths;
(b) flowing reactants through the tortuous flow paths;
(c) creating turbulent flow as the reactants flow through the tortuous flow paths;
(d) engaging the reactants with the catalyst material on the pore walls; and
(e) reacting the reactants to form a product.

12. The method according to claim 11 wherein the chemical reaction is a syngas reaction.

13. The method according to claim 11 wherein the chemical reaction is a Fischer-Tropsch reaction.

14. The method according to claim 11 wherein the chemical reaction is an oxidative dehydrogenation reaction.

15. The method according to claim 11 wherein the tortuous flow paths have a tortuosity greater than 1.0.

16. The method according to claim 11 wherein the catalyst bed comprises:
a plurality of monolith sections;
said monolith sections having a plurality of said pores extending therethrough; and
the pores of one monolith section being misaligned with the pores of an adjacent section to form said tortuous flow paths.

17. The method according to claim 11 wherein the catalyst bed comprises:
a plurality of monolith sections; and
at least one foreign material section disposed between adjacent monolith sections.

18. The method according to claim 16 wherein said foreign material section 19 selected from the group of wire gauze, wire mesh, foam or honeycomb material, wherein such shapes are formed using metal and/or metal oxide compositions, and metals are selected from the transition metal group of elements and metal oxides are selected from the group consisting of ceramic oxide, alkaline oxide and rare earth oxides.
